(19) United States
(12) Patent Application Publication
(10) Pub. No.: US 2014/0303023 A1
(43) Pub. Date: Oct. 9, 2014

(54) METHOD OF DIAGNOSING RENAL DISORDERS

(71) Applicant: Gert Mayer, Innsbruck (AT)

(72) Inventors: Gert MAYER, Innsbruck (AT); Michael RUDNICKI, Zirl (AT); Paul PERCO, Vienna (AT)

(21) Appl. No.: 14/258,891

(22) Filed: Apr. 22, 2014

Related U.S. Application Data
(63) Continuation of application No. 13/377,438, filed on Dec. 9, 2011, now abandoned.

(30) Foreign Application Priority Data
Jun. 9, 2009 (EP) 09162296.9

(51) Int. Cl. G01N 33/53; C12Q 1/68

(52) U.S. Cl. CPC G01N 33/53; C12Q 1/6883
USPC 506/9; 435/7.21; 435/6.12

(57) ABSTRACT

The invention refers to an in vitro method of determining the risk of renal disorders, in a patient, by measuring a VCAN parameter, characterized in that at least one of the isoforms V0 and V1 are specifically determined in a sample of said patient and compared to a reference level.

The present invention relates to a method for determining given renal disorders or the risk of developing renal disorders in a patient by measuring a VCAN parameter.

Renal disorders, also called nephropathies, are diverse, but individuals with kidney disease frequently display characteristic clinical features including the nephritic and nephrotic syndromes, acute kidney failure, chronic kidney disease, urinary tract infection, nephrolithiasis, and urinary tract obstruction.

Acute kidney injury (AKI) is in the clinical setting described as acute renal failure (ARF) or acute tubular necrosis (ATN) and refers to the spontaneous and significant decrease in renal function. AKI therefore reflects the entire spectrum of ARF, recognizing that an acute decline in kidney function is often secondary to an injury that causes functional or structural changes in the kidneys. ARF is a frequent and serious problem with a variety of adverse short- and long-term clinical consequences. Loss of function of the kidney, a vital organ, in the form of acute renal failure represents a special hazard, in particular to older patients, despite modern therapies including the use of the various forms of artificial kidney. In diagnosis and prognosis care must be taken to differentiate between functional renal insufficiency and intrinsic injury with morphologic damage.

AKI in particular in the intensive care unit is often associated with multiple organ failure and sepsis. Furthermore, AKI is associated with high mortality and morbidity in humans. Patients, for instance, experience AKI in ischemic reperfusion injury, along with treatment with nephrotoxic compounds including but not limited to antibiotics or anticancer drugs, application of contrast media e.g. when performing angiography resulting in nephropathy or nephrotoxicity, or at the intensive care unit, e.g. in the context of sepsis. The annual number of patients receiving contrast media is more than 100 million in the developed countries, and the rate of acute kidney injury ranges in a percent range, if coupled to risk factors like hypotension or diabetes.

AKI is usually categorised according to pre-renal, intrinsic and post-renal causes.

Pre-renal (causes in the blood supply):
hypovolemia (decreased blood volume), usually from shock or dehydration and fluid loss or excessive diuretics use.
hepatorenal syndrome, in which renal perfusion is compromised in liver failure
vascular problems, such as atheroembolic disease and renal vein thrombosis (which can occur as a complication of the nephrotic syndrome)
infection usually sepsis, systemic inflammation due to infection
severe burns
sequestration due to pericarditis and pancreatitis
hypotension due to antihypertensives and vasodilators

Intrinsic (damage to the kidney itself):
toxins or medication (e.g. some NSAIDs, aminoglycoside antibiotics, iodinated contrast, lithium, phosphate nephropathy due to bowel preparation for colonoscopy with sodium phosphates)
rhabdomyolysis (breakdown of muscle tissue)—the resultant release of myoglobin in the blood affects the kidney; it can be caused by injury (especially crush injury and extensive blunt trauma), statins, stimulants and some other drugs
hemolysis (breakdown of red blood cells)—the hemoglobin damages the tubules; it may be caused by various conditions such as sickle-cell disease, and lupus erythematosus
multiple myeloma, either due to hypercalcemia or “cast nephropathy” (multiple myeloma can also cause chronic renal failure by a different mechanism)
acute glomerulonephritis which may be due to a variety of causes, such as anti glomerular basement membrane disease/Goodpasture's syndrome, Wegener's granulomatosis or acute lupus nephritis with systemic lupus erythematosus

Post-renal (obstructive causes in the urinary tract) due to:
medication interfering with normal bladder emptying (e.g. anticholinergics).
benign prostatic hypertrophy or prostate cancer.
kidney stones.
due to abdominal malignancy (e.g. ovarian cancer, colorectal cancer).
obstructed urinary catheter.
drugs that can cause crystalluria and drugs that can lead to myoglobinuria and cystitis

According to the state of the art, renal failure is diagnosed when either creatinine or blood urea nitrogen tests are markedly elevated in an ill patient, especially when oliguria is present. Previous measurements of renal function may offer comparison, which is especially important if a patient is known to have chronic renal failure as well. If the cause is not apparent, a large amount of blood tests and examination of a urine specimen is typically performed to elucidate the cause of acute renal failure, medical ultrasonography of the renal tract is essential to rule out obstruction of the urinary tract.

An exemplary consensus criterium for the diagnosis of AKI is at least one of the following:
Risk: serum creatinine increased 1.5 times or urine production of less than 0.5 ml/kg body weight for 6 hours
Injury: creatinine 2.0 times OR urine production less than 0.5 ml/kg for 12 h
Failure: creatinine 3.0 times OR creatinine more than 355 μmol/l (with a rise of more than 44) or urine output below 0.3 ml/kg for 24 h
Loss: persistent AKI or complete loss of kidney function for more than four weeks
End-stage Renal Disease: complete loss of kidney function for more than three months.

A rapid increase in serum creatinine may also be an indicator for a high AKI risk following medical treatment, e.g. impairment in renal function is indicated by an increase in serum creatinine by more than 0.5 mg/dl or more than 25% within 3 days after medication.

Kidney biopsy may be performed in the setting of acute renal failure, to provide a definitive diagnosis and sometimes an idea of the prognosis, unless the cause is clear and appropriate screening investigations are reassuringly negative.

To diagnose AKI, usually urine and blood tests are done and the volume of urine produced is monitored.

The gold standard for diagnosing AKI is the measurement of serum creatinine. Unfortunately, creatinine as marker has several limitations. On the one hand, levels of serum creatinine widely vary among individuals depending on age, sex, muscle mass or medication status. On the other hand, serum creatinine does not accurately depict kidney function during acute changes in glomerular filtration as it is a marker, which can only be interpreted in steady state. Furthermore creatinine levels do not rise until damage is severe and kidney function already declines. Other biomarkers such as lipocalin 2 (LCN2), also known as NGAL (neutrophil gelatinase-associated lipocalin), kidney injury molecule 1 (KIM1), cysteine-rich angiogenic inducer 61 (CYR61), or interleukin 18 (IL18) have recently been proposed as alternative parameters for the detection of acute kidney injury.

Patients with normal kidney function are currently not tested for any renal disease biomarkers. In the absence of any functional kidney disorder, such as urine volume reduction or creatinine level, it is assumed that there is no risk for developing AKI. However, there are patients, who have the potential to develop AKI upon certain medical treatment, which could be damaging to the kidney function, such as simple radiography using a contrast medium or chemotherapy. Several risk factors for acute renal failure have been identified so far.

High-risk patients are considered those with chronic diseases that can affect the kidneys like diabetes, hypertension and heart disease. Pregnant patients who suffer from eclampsia, a hypertensive condition, also have a high risk for kidney damage.

Some drugs are nephrotoxic, i.e. poisonous to the kidney, and therefore damaging to the kidneys. This includes certain antibiotics like aminoglycosides, anti-inflammatory drugs and the contrast media used in specific X-ray tests of the urinary tract. A need therefore exists for a marker which can be used to specifically and reproducibly detect the presence of, or predisposition to acquiring AKI clinically leading to ARF.

Chronic kidney disease (CKD) affects up to 13% of the general population and its prevalence is steadily rising. Progressive loss of kidney function is accompanied by increased morbidity and mortality from cardiovascular disease and bone metabolism disorders, and the treatment of end-stage renal disease is a major healthcare challenge. Since the natural history of CKD shows a high intraindividual variation reliable histological and serological markers capable to differentiate between stable and progressive disease are heavily needed. Published data on biomarkers predicting progression are scarce, and their significance is often limited.

The increasing prevalence of patients on renal replacement therapy has become a major challenge for healthcare systems. Frequently, end stage renal disease (ESRD) is the terminal phase of a chronic process. A better understanding of the pathophysiology of progressive kidney disease could lead to the development of new treatment options which might be able to stabilize renal function and reduce the incidence of ESRD. In order to use new but also the already available drugs even more efficiently, it is also highly desirable to identify patients with an adverse renal prognosis in the early phases of the disease as not all subjects show a relentlessly progressive decline in renal function. In this context the magnitude of proteinuria has been suggested to be a useful risk marker, even though, on an individual basis, the discriminatory power is questionable. Other biomarkers such as apolipoprotein A-IV (APOA4), adiponectin (ADIPOQ), or fibroblast growth factor 23 (FGF23) have recently been proposed as alternative parameters to predict the course of disease.

Cardiovascular disease (CVD) is a major cause of morbidity and mortality in patients suffering from chronic kidney disease. Around 50% of deaths of patients with end-stage renal disease are caused by cardiovascular complications. At the same time almost all patients with ESRD show signs of renal osteodystrophy, a heterogeneous pattern of bone metabolism disorders caused by chronic renal insufficiency and concomitant diseases.

The elevated risk of CVD in chronic kidney patients is partly based on traditional risk factors such as hypertension or diabetes mellitus. Next to these traditional risk factors a number of biomarker candidates are discussed in the scientific literature to be predictive for cardiovascular outcomes in patients with chronic kidney disease although none is used in the routine diagnostics so far. These marker candidates are involved in processes of inflammation, oxidative stress, or vascular calcification among others.

Several proteins have been identified as molecular biomarker candidates of kidney damage. The clinical significance of these heterogeneous biomarkers is rather difficult to compare due to the variety of clinical settings in which they have been tested such as AKI, diabetic- and non-diabetic CKD, polycystic kidney disease, and dysfunction of kidney grafts. However, their predictive power for progressive decline of kidney function has not been tested in all cases. Moreover, the expression of some of these markers is not kidney specific or restricted to the kidney, and therefore their levels can be influenced by certain non-renal pathologies such as cardiovascular disease, diet, or concomitant medication.

Rudnicki et al (Nephron Exp Nephrol 2004; 97:e86-e95) describe gene expression analysis of a human kidney cell line using cDNA microarrays, and a correlation between microarray and qRT-PCR results.

Rudnicki et al (Kidney International 2007, 71, 325-335) disclose the gene expression profiles of human proximal tubular epithelial cells in proteinuric nephropathies. 168 different genes have been characterized.

Perco et al (European Journal of Clinical Investigation (2006) 36, 753-763) describe protein biomarkers associated with acute renal failure and chronic kidney disease.

Biomarkers indicative for progressive disease are described in PCT/EP2008/068083. Such biomarkers are selected from the group consisting of IL1RN, ISG15, LIFR, C6 and IL32.

Versican is described as an AKI risk factor by PCT/EP2009/054439.

WO2007/096142A2 describes vascular tumor markers, among them versican, and a method for identifying diseases associated with neovascularisation.

Stokes et al (Kidney International 59(2) 532-542, 2001) describe a pathogenic role for versican in crescentic glomerulonephritis (CGN). Renal tissues from CGN patients are immunohistochemically examined for versican, using rabbit polyclonal antibody directed to human versican (VC-E).

WO2009/061368 describes inhibition of versican and antibodies against versican.

Dours-Zimmermann et al (The Journal of Biological Chemistry 269(52) 32992-32998, 1994) disclose the determination of isoforms V0 and V1 in a non-differentiated way, using RT-PCR and immunoblot detection.

Cattaruzza et al (Journal of Biological Chemistry 277(49) 47626-47635, 2002) have carried out a molecular mapping of distributions of PG-M/ versican isoforms V0-V3 in human tissues and investigated how the expression of these isoforms is regulated in endothelial cells in vitro.

Arslan et al (British Journal of Cancer 96(10) 1560-1568, 2007) describe the increased expression of certain versican isoforms in the extracellular matrix, which plays a role in tumor cell growth, adhesion and migration.

WO91/08230 describes antibodies against the NH2-terminal domain or glycosaminoglycan attachment domain of versican.

It is a goal of the present invention to provide a universal marker specifically indicative for renal disorders.

The object is solved by the method according to the invention, which provides for the in vitro determination of the risk of renal disorders in a patient, by measuring a VCAN parameter or a parameter, which is related to VCAN, characterized in that at least one of the isoforms V0 and V1 are specifically determined in a sample of said patient and compared to a reference level. The term “risk of renal disorders” include any kind of renal disorders, the risk of developing renal disorders or the risk of a progressive renal disorder. Determining the risk of renal disorders shall mean the risk assessment as well as determining renal disease, including its diagnosis, prognosis, progression, monitoring and influence of test compounds or therapeutics on such disease.

The term “specific” determination or “specifically” determining with respect to the method according to the invention refers to a reaction of a reagent that is determinative of the versican isoform of interest in a population of molecules comprising the versican isoform of interest and at least one further versican isoform. Thus, under designated assay conditions, the reagent binds to its particular target isoform and does not bind in a significant amount to another isoform or other molecules present in a sample. The specific determination in particular means that the readout is selective in terms of the individual target identity, thus differentiating from other, similar targets, such as other isoforms. The selective determination is usually achieved, if the target recognition is at least 3 fold different, preferably at least 5 fold different, preferably at least 10 fold different, preferably the difference is at least 100 fold, and more preferred a least 1000 fold.

The preferred method according to the invention relates to the specific determination of both isoforms, i.e. the determination of both isoforms on an individual basis, in the same or the same type of sample.

It was surprisingly found that employing the inventive isoforms of versican a specific risk determination of renal disorders in general is feasible. Preferably said renal disorders are selected from acute, diabetic- and non-diabetic chronic, polycystic, proteinuric or progressive kidney disease, dysfunction of kidney grafts, and associated increased morbidity or mortality from cardiovascular disease and bone metabolism disorders. The method according to the invention would, however, preferably exclude the determinion of renal cancer or tumors.

In particular, by the method according to the invention a renal disease is determined, such as disorders selected from IgA nephropathy, non IgA mesangioproliferative glomerulonephritis, membranoproliferative glomerulonephritis, any postinfectious glomerulonephritis, focal-segmental glomerulosclerosis, minimal change disease, membranous nephropathy, lupus nephritis of any kind, vasculitides with renal involvement of any kind, any other systemic disease leading to renal disease including but not being limited to diabetes mellitus, hypertension or amyloidosis, any hereditary renal disease, any interstitial nephritis and renal transplant failure.

In a preferred method according to the invention the amount of said parameter is increased at least 1.5 times the reference value of subjects not at risk of the renal disorder.

The preferred method comprises sampling from the patient's tissue or body fluid, such as to provide a sample, which is a tissue, blood, serum, plasma or urine sample. In particular, the sample preferably used is a kidney biopsy sample.

The determination method preferably comprises the determination of the VCAN expression, either one of the inventive isoforms or both. The VCAN expression is preferably determined as VCAN nucleic acid and/or protein expression.

A preferred method according to the invention relates to the determination of a respective parameter by microarray hybridization with specific probes or by PCR.

In a preferred method the inventive parameter is tested in combination with a further kidney risk factor (KRF) or senescence parameter. Preferably combined KRF are markers selected from the group consisting of IL1RN, ISG15, LIFR, C6, IL32, NRP1, CCL2, CCL19, COL3A1 and GZMM. Other combinations of any of the inventive versican isoforms with each other or any other relevant biomarker associated with renal disorders and related conditions would be feasible.

Preferably combined senescence parameters are selected from the group consisting of chronological age, telomere length, CDKN2A and CDKN1A. Other senescence parameters commonly used to determine a correlation with chronological age may be employed as well, such as those, which are either regulators of p53, associated with DNA repair, cell cycle control, telomere binding and cell surface remodelling. Exemplary senescence associated genes are selected from the group consisting of Sirtiuns 1-8, XRCC5, G22P1, hPOT 1, Collagenase, TANK 1,2, TRF 1,2 and WRN.

According to the invention there is preferably provided a method for the diagnosis or prognosis of progressive proteinuric kidney disease, renal disease in a patient at risk of disease progression or kidney failure.

A specific aspect of the invention refers to a set of reagents and the use of such set for determining the risk of renal disorders, comprising
a reagent specifically binding to VCAN V0 polypeptide, and
a reagent specifically binding to VCAN V1 polypeptide.

In particular, the reagents differentiate between VCAN0 and VCAN1 polypeptides. Either a mixture of the reagents or a set of single components may be provided. The set according to the invention preferably employs reagents, which are antibodies or antibody fragments, preferably monoclonal antibodies specifically recognizing one of the inventive isoforms. Preferably reagents as used in a set according to the invention are used together with detection means, such as a label. Preferred reagents are labelled.

Therefore, the present invention provides a method of determining renal disorders, which is particularly important for determining a progressive disease, e.g. the risk of disease conditions terminally associated with end-stage renal failure. A method for diagnosing a progressive disease and/or assessing long term prognosis of a disease would provide for qualifying high risk patients early on, even before the diagnosis of a chronic disease.

It has been surprisingly found out that the versican V0 and V1 isoforms or splice variants are specifically determinative of high risk patients. Unexpectedly, the expression of the individual isoforms V0 and V1 turned out to be significantly higher in patients with a progressive clinical course of disease. Other isoforms like V2 and V3 did not correlate with renal disorders, such as progressive disease. For the inventive method one of these markers or associated parameters can be detected, which relate to the specific markers with a high correlation.

Versican (VCAN-UniGene: Hs.643801, Hs.715773, GeneID: 1462, GenBank: AA056022/AA056070) is a major extracellular chondroitin sulfate proteoglycan also known as Chondroitin sulfate proteoglycan core protein 2 (CSPG-2), PG-M, or Chondroitin sulfate proteoglycan 2.

VCAN V0, also called VCAN0, is a specific isoform, the transcript variant 1, which corresponds to the longest isoform. The protein sequence is retrieved from the International Protein Index (IPI), a database hosted by the European Bioinformatics Institute (EBI) http://www.ebi.ac.uk/IPI/IPIhelp.html. The sequence of isoform V0 of versican core protein is provided as SEQ ID No: 1.

The VCAN0 mRNA sequence is retrieved from the NCBI nucleotide database.

http://www.ncbi.nlm.nih.gov/. The sequence is listed in SEQ ID No. 2

VCAN V1, also called VCAN1, has a shorter sequence than VCAN0. The protein sequence is retrieved from the International Protein Index (IPI), a database hosted by the European Bioinformatics Institute (EBI) http://www.ebi.ac.uk/IPI/IPIhelp.html. The sequence of isoform V1 of versican core protein is provided as SEQ ID No: 3.

The term“VCAN0 and/or VCAN1” as used herein shall refer to markers, including but not limited to respective polypeptides and nucleotide sequences, such as native-sequence polypeptides, chimeric polypeptides, a derivative, an essential part of the splice variants, and precursors thereof, and modified forms of the polypeptides and derivatives, or nucleic acids encoding such polypeptides, which may be included in a biological sample, are referred to herein as inventive isoforms or inventive markers.

Increased expression of the hyaluronan-binding proteoglycan versican was found to be associated with (i) age, (ii) serum creatinine at time of biopsy in diabetic nephropathy, (iii) progressive decline of renal function in proteinuric nephropathies and (iv) acute tubular injury, tubular atrophy and interstitial fibrosis in zero-hour kidney transplant biopsies. When the expression of VCAN was evaluated, it was surprisingly found that the isoforms V0 and V1, but not V2 and V3, were appropriate markers to determine progressive disease. By an exemplary method according to the invention it was found that the expression of the isoforms V0 and V1 was significantly higher in patients with progressive disease (V0: 3.7 fold, p=0.0025; V1: 2.1 fold, p=0.014). The isoform V2 was not expressed in these samples, and no differences of the expression of the isoform V3 between stable and progressive subjects was found. In an extended study these results have been confirmed. To evaluate which cells in the kidney might contribute to VCAN expression, the basal expression of all VCAN isoforms was determined in vitro. VCAN isoforms V0 and V1 were highly expressed in various epithelial tubule cell lines and in skin fibroblasts, but to a much lesser extent in foreskin fibroblasts, prostate epithelial cells, smooth muscle cells and colon carcinoma cells. Versican has also been determined by immunohistochemistry in human kidney biopsies. Versican mRNA was determined in a mouse model of glomerulonephritis. The differentiation of the versican isoforms according to the inventive method will provide for the improved determination of renal disorders. The in vitro results particularly suggested a cell specific and an organ specific expression of VCAN V0 and V1 isoforms in the kidney.

As a read out, the amount of parameters in a sample to determine the inventive VCAN markers may be measured and correlated to the risk of said patients, which can be low, medium or high, or else prediction rules established in order to discriminate between the binary outcome stable or progressive disease. For example, the ability of a prediction rule can be assessed by calculating the area under the ROC curve (AUC) using the Sommer's D statistic. The relation between the area under the ROC and Sommer's D is the following:

AUC=(1+Sommer's D)/2.

It is preferred to employ a marker according to the invention either as single predictor of progression with an AUC value of at least 0.5, preferably at least 0.6, more preferred 0.7, 0.8 or even at least 0.9. Preferred marker combinations reach AUC values of at least 0.6, preferably at least 0.7, 0.8 or even at least 0.9, up to 1.0.

With reference to a healthy patient or a stable disease patient, the preferred method according to the invention qualifies a significant risk when an increase of single parameters by at least 50%, preferably at least 60%, more preferred at least 70%, more preferably at least 80%, more preferably at least 100% is determined.

The high risk progressive nature of the disease is preferably indicated, if the amount of a marker or the combination of markers is increased at least 1.5 times the reference value of subjects not suffering from the progressive disease, preferably being healthy subjects or subjects suffering from a chronic non-progressive disease.

In special embodiments the amount of VCAN0 or VCAN1 is at least 1.5, preferably at least 1.6, at least 1.8, at least 2, at least 3, at least 4, at least 5, at least 6, or at least 8 times the reference value, in particular as determined by PCR with either PPIA or GAPDH as endogenous controls or as determined by microarray analysis.

If more than one marker is detected, the comparison is made to each single reference value for each marker in the non-progressive disease or healthy reference itself.

The inventive method can distinguish if a chronic disease is stable, i.e. the symptoms do not significantly increase over a period of about at least or up to four, six, eight, ten months, one, two or three years after the sample was obtained, or is a progressive disease, i.e. the condition of the subject will increasingly suffer, e.g. over the same time span.

Patients at risk of a renal progressive disease have an increased risk of gradual worsening of renal disease.

The National Kidney Foundation's Kidney Disease Outcomes Quality Initiative (NKF KDOQI) classified chronic kidney disease (CKD) into five stages with stage five indicating terminal kidney failure. Stage 1 patients have kidney damage with normal glomerular filtration rate (GFR) values above 90 ml/min/1.73 m2. Patients in stage two have slightly decreased GFR values between 60 and 89 ml/min/1.73 m2. Stage three patients have moderately decreased GFR values between 30 and 59 ml/min/1.73 m2. Patients in stage four experience severely decreased GFR values between 15-29 ml/min/1.73 m2. Kidney failure, also defined as end-stage renal disease, is reached in stage five when patients have GFR values lower than 15 ml/min/1.73 m2. End-stage renal disease is followed by renal replacement therapy with the treatment options dialysis or organ transplantation.

If the risk of end-stage renal failure is high, the disease stages will be passed very quickly, which would result in the need for kidney dialysis and transplantation. To delay the terminal phase of renal disease a patient which was diagnosed as having an increased risk of disease progression would receive the appropriate medication early on employing aggressive treatment regimens.

The risk of a patient to suffer from kidney or renal disease progression may be diagnosed at an early stage of disease, even before a chronic disease has been diagnosed. On the other hand a prognosis is provided, which would quantify the fast progression of the disease in a patient already suffering from chronic renal disease.

Thus, the inventive method can include the step of obtaining the sample from a patient potentially suffering from a progressive renal disease, where a chronic renal disease may already have been diagnosed or not. The method according to the invention is preferably employed with a kidney biopsy sample, such as wedge or needle sample, or else from tubular cells, and also by detecting the markers in serum, blood, plasma and urine by comparing reference values of standard values or from healthy subjects.

The term “patients” herein includes subjects suffering from or at risk of renal disorders, but also healthy subjects. The subject can, e.g., be any mammal, in particular a human, but also selected from mouse, rat, hamster, cat, dog, horse, cow, pig, etc. The inventive method can also include the step of obtaining the sample from a patient at risk for developing acute kidney injury, e.g. before contrast medium administration in the course of angiography.

The invention also provides a method of assessing whether a patient is at risk of a renal disorder, comprising comparing:

(a) levels of the V0 and/or V1 isoform(s) in a sample from said patient, and

(b) normal levels of said isoform(s) in samples of the same type obtained from control patients, wherein altered levels of the isoform(s) relative to the corresponding normal levels is an indication that the patient has a risk of renal disorder, e.g. a predisposition to kidney disease, such as AKI or disease progression, in particular where detection of a level of an isoform that differs significantly from the standard indicates acute kidney disease or onset of kidney disease or increased risk for developing ARF or disease progression. A significant difference between the levels of an inventive isoform in a patient and the normal levels is an indication that the patient has a risk of kidney disease or a predisposition to kidney disease.

It is explicitly understood that the method according to the invention is carried out in vitro, including ex vivo settings.

The inventive markers can be detected in any sample of a subject comprising said markers e.g. where an expression of an inventive isoform is determined either as polynucleotide, e.g. as mRNA, or expressed polypeptide or protein. The comparison with the reference should be of the same sample type. The comparison with the reference value should be of the same sample type. In particular, the sample can be tissue, e.g. of a biopsy, blood, serum, plasma or a urine sample.

Reference values for the inventive isoforms are preferably obtained from a control group of patients or subjects with normal expression of said isoform, or an isoform expression, that is afflicted with kidney stress conditions, such as septic, cancer or diabetic patients, without proteinuremia or AKI, which represents the appropriate reference patient group. In a particular aspect, the control comprises material derived from a pool of samples from normal patients.

The term “detect” or “detecting” includes assaying, imaging or otherwise establishing the presence or absence of the target versican isoform encoding the markers, subunits thereof, or combinations of reagent bound targets, and the like, or assaying for, imaging, ascertaining, establishing, or otherwise determining one or more factual characteristics of kidney disease or similar conditions. The term encompasses diagnostic, prognostic, and monitoring applications for an inventive versican isoform.

In preferred embodiments, determining the amount of the inventive marker or any combination thereof comprises determining the expression of the marker(s), preferably by determining the mRNA concentration of the marker(s). To this extent, mRNA of the sample can be isolated, if necessary after adequate sample preparation steps, e.g. tissue homogenisation, and hybridized with marker specific probes, in particular on a microarray platform with or without amplification, or primers for PCR-based detection methods, e.g. PCR extension labelling with probes specific for a portion of the marker mRNA. In preferred embodiments the marker(s) or a combination thereof is (are) determined by microarray hybridization with VCAN0 and/or VCAN1 specific probes, or by PCR.

Differential expression of the polynucleotides is preferably determined by microarray, hybridization or by amplification of the extracted polynucleotides. The invention contemplates a gene expression profile comprising one or both of the inventive markers. This profile provides a highly sensitive and specific test with both high positive and negative predictive values permitting diagnosis and prediction of the patient's risk of developing disease.

For example, the invention provides a method for determining the risk of renal disorders in a patient comprising

(a) contacting a sample obtained from said patient with oligonucleotides that specifically hybridize to the V0 and/or V1 isoform(s), and

(b) detecting in the sample a level of polynucleotides that hybridize to the isoform(s) relative to a predetermined cut-off value, and therefrom determining the risk of renal disorders in the subject.

Within certain preferred embodiments, the amount of polynucleotides that are mRNA are detected via polymerase chain reaction using, for example, oligonucleotide primers that hybridize to an inventive isoform, or complements of such polynucleotides. Within other embodiments, the amount of mRNA is detected using a hybridization technique, employing oligonucleotide probes that hybridize to an inventive isoform, or complements thereof. When using mRNA detection, the method may be carried out by combining isolated mRNA with reagents to convert to cDNA according to standard methods and analyzing the products to detect the presence of the isoform in the sample.

In particular aspects of the invention, the methods described herein utilize one or both inventive markers placed on a microarray so that the expression status of each of the markers is assessed simultaneously. In an embodiment, the invention provides a microarray comprising a defined set of marker genes, whose expression is significantly altered by a risk of renal disorders. The invention further relates to the use of the microarray as a prognostic tool to predict kidney disease.

In further embodiments the amount of a marker or any combination thereof is determined by the polypeptide or protein concentration of the marker(s), e.g. with marker specific ligands, such as antibodies or specific binding partners. The binding event can, e.g., be detected by competitive or non-competitive methods, including the use of labelled ligand or marker specific moieties, e.g. antibodies, or labelled competitive moieties, including a labelled marker standard, which compete with marker proteins for the binding event. If the marker specific ligand is capable of forming a complex with the marker, the complex formation indicates expression of the markers in the sample.

In particular, the invention relates to a method for diagnosing and/or monitoring renal disorders in a patient by quantitating the V0 and/or V1 isoform(s) in a sample from the subject comprising

(a) reacting the sample with one or more binding agents specific for the isoform(s), e.g. an antibody that is directly or indirectly labelled with a detectable substance, and

(b) detecting the detectable substance.

VCAN isoform levels can be determined by constructing an antibody microarray, in which binding sites comprise immobilized, preferably monoclonal antibodies specific to a marker. The invention also relates to kits for carrying out the methods of the invention.

The invention further contemplates the methods, compositions, and kits described herein using additional markers associated with kidney disease. The methods described herein may be modified by including reagents to detect the additional markers, or polynucleotides for the markers.

Appropriate probes, specific antibodies or methods for determining the biomarkers are known in the art, and have been used for different purposes.

In general, immunoassays involve contacting a sample containing or suspected of containing a biomarker of interest with at least one antibody that specifically binds to the biomarker. A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing immunoassays. But any suitable immunoassay may be utilized, for example, enzyme-linked immunoassays (ELISA), radioimmunoassays (RIAs), competitive binding assays, and the like.

Antibodies or other polypeptides may be immobilized onto a variety of solid supports for use in assays. Solid phases that may be used to immobilize specific binding members include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Biological assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In exemplary embodiments, the analyte is measured using standard sandwich enzyme immunoassay techniques. A first antibody which binds the analyte is immobilized in wells of a 96 well polystyrene microplate. Analyte standards and test samples are pipetted into the appropriate wells and any analyte present is bound by the immobilized antibody. After washing away any unbound substances, a horseradish peroxidase-conjugated second antibody which binds the analyte is added to the wells, thereby forming sandwich complexes with the analyte (if present) and the first antibody. Following a wash to remove any unbound antibody-enzyme reagent, a substrate solution comprising tetramethylbenzidine and hydrogen peroxide is added to the wells. Color develops in proportion to the amount of analyte present in the sample. The color development is stopped and the intensity of the color is measured at 540 nm or 570 nm. An analyte concentration is assigned to the test sample by comparison to a standard curve determined from the analyte standards.

Marker specific moieties are substances which can bind to or detect at least one of the markers for a detection method described above and are in particular marker nucleotide sequence detecting tools or marker protein specific antibodies, including antibody fragments, such as Fab, F(ab), F(ab)′, Fv, scFv, or single chain antibodies. The marker specific moieties can also be selected from marker nucleotide sequence specific oligonucleotides, which specifically bind to a portion of the marker sequences, e.g. mRNA or cDNA, or are complementary to such a portion in the sense or complementary anti-sense, like cDNA complementary strand, orientation.

For easy detection the moieties are preferably labelled, such as by optical, including fluorescence, and radioactive labels.

The inventive prognosis method can predict whether a patient is at risk of developing acute kidney injury. The higher the fold increase, the higher is the patient's risk of AKI. An elevated level of an inventive isoform indicates, for example, special treatment of the patient, using appropriate medication or contrast media. The method of the invention can thus be used to evaluate a patient before, during, and after medical treatment.

Likewise, the inventive isoform level can be compared to a cut-off concentration and the kidney disease development potential is determined from the comparison; wherein concentrations of the versican isoform above the reference concentrations are predictive of, e.g., correlate with, kidney disease development in the patient.

Thus, the preferred method according to the invention comprises the step of comparing the KRF level with a predetermined standard or cut-off value, which is preferably at least 50% higher than the standard, more preferred at least 60% or 70% higher, but can also be at least 100% higher.

In aspects of the methods of the invention, the methods are non- or minimally invasive for renal disorders predisposition testing, which in turn allow for diagnosis of a variety of conditions or diseases, e.g. associated with acute kidney disease. In particular, the invention provides a non-invasive non-surgical method for detection, diagnosis, monitoring, or prediction of acute kidney disease or onset of kidney disease in a patient comprising: obtaining a sample of blood, plasma, serum, urine or saliva or a tissue sample from the patient; subjecting the sample to a procedure to detect one or both of the inventive isoforms by comparing the levels of the isoform to the levels of the isoform obtained from a control.

The invention also contemplates a method of assessing the potential of a test compound to contribute to kidney disease or onset of kidney disease comprising:

(a) maintaining separate aliquots of a sample from a patient in the presence and absence of the test compound, and

(b) comparing the levels of the V0 and/or V1 isoform(s) in each of the aliquots.

This is particularly useful in monitoring the versican isoform level in clinical trials. A significant difference between the levels of an inventive isoform in an aliquot maintained in the presence of or exposed to the test compound relative to the aliquot maintained in the absence of the test compound, indicates that the test compound potentially contributes to kidney disease or onset of kidney disease.

Likewise, the invention can be employed to determine the effect of an environmental factor on kidney disease comprising comparing one or both of the inventive isoforms associated with kidney disease or onset of kidney disease in the presence and absence of the environmental factor.

In a further aspect the present invention provides a set that contains or consists of at least two different reagents or marker specific moieties, to specifically determine both of the inventive VCAN variants on an individual basis. Besides, further markers may be determined in the same sample for the same or a different purpose.

Marker specific moieties used as preferred reagents in such a set according to the invention are substances which can bind to or detect at least one of the markers for a detection method described above and are in particular marker protein specific antibodies or antibody fragments, such as Fab, F(ab)2, F(ab)′, Fv, scFv, or single chain antibodies. The marker specific moieties can also be selected from marker nucleotide sequence specific oligonucleotides, which specifically bind to a portion of the marker sequences For easy detection the moieties are preferably directly or indirectly labelled, such as by optical, including fluorescence, and radioactive labels.

### FIGURES

FIG. 1. Correlation of versican isoform V0 RNA levels with eGFR at time of biopsy (A) and with eGFR at latest follow up (B), and of V1 RNA levels with eGFR at time of biopsy (C) and with eGFR at latest follow up (D). eGFR estimated glomerular filtration rate in ml/min/1.73 m2

FIG. 2. Versican isoform expression in stable and progressive kidney diseases.

FIG. 3. Expression of versican mRNA in vitro. The basal expression of versican isoforms was measured in various renal and non-renal cell lines. K2 primary proximal tubule cells, HK2 and hTERT-RPTC immortalized renal proximal tubule cells, HF primary skin fibroblasts, VHF primary foreskin fibroblasts, SMC primary smooth muscle cells, EP immortalized prostate epithelial cells, CACO2 colon carcinoma cells, LLC-PK1 pig renal tubule cells, kidney: whole kidney tissue. The expression values are shown as ratio to PPIA.

FIG. 4. VCAN expression in a mouse model of glomerulonephritis.

FIG. 5. Versican protein expression in renal disease.

Versican protein expression was detected in representative stable and progressive subjects (arrows). Versican protein was expressed both in the glomerular (A) and in the tubulointerstitial (B) compartment, however, expression was more prominent at the tubular basal membrane (B) and in the interstitiu (C). Furthermore, versican was also detected in the media of renal cortical blood vessels (D).

The present invention is further illustrated by the following figures and examples without being limited thereto.

### EXAMPLES

The incidence and prevalence of chronic kidney disease (CKD) is increasing worldwide and has been predicted to soon reach epidemic proportions. Chronic kidney diseases is caused by primary renal diseases such as IgA nephropathy (IgAN), minimal change disease (MCD), focal-segmental glomerulosclerosis (FSGS), membranous nephropathy (MN) and membranoproliferative glomerulonephritis (MPGN), as well as by systemic diseases (e.g. systemic lupus erythematodes, diabetes mellitus type 1 and type 2, or hypertension), which can also lead to deterioration of kidney function. In a proportion of these patients CKD progresses to end-stage renal disease (ESRD) which requires renal replacement therapy such as dialysis and kidney transplantation. These therapies represent a major challenge for healthcare systems. But even slight impairment of kidney function—far from ESRD-correlate with serious health consequences such as increased cardiovascular morbidity and mortality (e.g. myocardial infarction, sudden cardiac death, peripheral arterial disease), increased risk of pathological bone fractures due to renal osteodystrophy, and consequently with reduced quality of life.

To date only few general risk factors for the progression of renal failure have been firmly established. It is known that elevated serum creatinine at time of biopsy, hypertension, and the degree of proteinuria (typically >500-1000 mg/day) correlate with an unfavourable prognosis in various glomerulopathies. Although these clinical findings are of stronger predictive value, certain histopathological changes on kidney biopsies have also been associated with increased risk of progression. The degree of tubular atrophy and interstitial fibrosis is a better predictor of long-term renal survival than the extent of glomerular damage in almost all glomerular renal diseases including IgA nephropathy (IgAN), membranous nephropathy (MN), membranoproliferative glomerulonephritis (MPGN) and lupus nephritis (LN).

Herein the VCAN isoforms V0 and V1 were found to be novel biomarkers for adverse outcome in CKD.

Materials and Methods

Isoforms of Versican.

Five isoforms of versican (GeneID 1462) are listed in the International Protein Index (IPI) as given in the table below. Four of these isoforms (V0, V1, V2 and V3) are confirmed splice variants of the versican gene. The isoform Vint has been proposed as another isoform, which largely resembles isoform V0 and differs solely by a deletion/insertion in the carboxyterminal end of the RNA.

TABLE 1
VCAN isoforms as listed in the International Protein Index

| IPI | Accession | Description | SeqLength |
|---|---|---|---|
| IPI:IPI00009802.1 | IPI00009802 | Isoform V0 of versican | 3396 |
| | | core protein | |
| IPI:IPI00215628.1 | IPI00215628 | Isoform V1 of versican | 2409 |
| | | core protein | |
| IPI:IPI00215629.1 | IPI00215629 | Isoform V2 of versican | 1642 |
| | | core protein | |
| IPI:IPI00215630.1 | IPI00215630 | Isoform V3 of versican | 655 |
| | | core protein | |
| IPI:IPI00215631.1 | IPI00215631 | Isoform VINT of versican | 3370 |
| | | core protein | |

### Example 1

### Patients and Kidney Biopsies

In a first setting, we used 37 kidney biopsies obtained from patients with proteinuric renal diseases during their routine diagnostic workup for which we had complete clinical follow-up data (Table 2): diabetic nephropathy n=2, hypertensive nephropathy n=2, IgA nephropathy n=11, minimal change disease n=8, membranous nephropathy n=7, primary focal-segmental glomerulonephritis n=6, unknown n=1). The median follow-up time was 25 months (2-80). Based upon the estimated glomerular filtration rate (eGFR), which was calculated using the modified MDRD formula, patients were divided into a stable and a progressive cohort: Patients were defined stable when eGFR was >60 ml/min/1.73 m² at both timepoints, or when eGFR was <60 ml/min/1.73 m² at either timepoint and no decline in eGFR over time was observed. Patients were defined as progressive when eGFR was >60 ml/min/1.73 m² at time of biopsy and <60 ml/min/1.73 m² during follow-up, or when eGFR<60 ml/min/1.73 m² at both timepoints and delta eGFR was less than −1 ml/min/1.73 m², or when they reached end-stage renal disease. Tubular atrophy and interstitial fibrosis (TAIF) were scored by an independent pathologist following a semiquantitative grading system on haematoxylin/eosin and periodic-acid-Schiff- or Pearse-stained sections: none, mild (0-10%), moderate (11-30%), severe (>30%). The use of surplus material from routine biopsies for gene expression profiling has been accredited by the Institutional Review Board of the Medical University of Innsbruck.

RNA Isolation and Real-Time PCR.

Total RNA of whole kidney cryosections was isolated using the RNeasy® Micro Kit (Qiagen, Valencia Calif.). RNA was reverse transcribed into cDNA with the High Capacity cDNA reverse Transcription kit (Applied Biosystems, Foster City Calif.) in a 50 μl reaction according to the manufacturer's instructions. Preamplification was performed using TaqMan® Gene Expression Assays (vide infra) and the TaqMan® PreAmp Master Mix. Briefly, equal volumes of 20× TaqMan Gene Expression Assays were pooled and diluted to 0.2× with TE buffer. A 50 μl reaction containing 12.5 μl pooled assay mix, 25 μl TaqMan Preamp Master Mix and 5 ng of cDNA was prepared per sample and incubated in a thermocycler for 10 min at 95° C. followed by 10 cycles of 95° C. for 15 seconds and 60° C. for 4 minutes. Samples were then immediately cooled and diluted to 250 μl with TE buffer. All gene expression assays used had been previously tested to ensure uniform preamplification as recommended by the manufacturer.

The preamplified cDNA was analysed on the 7500 Fast Real-Time PCR System (Applied Biosystems) using the following inventoried TaqMan® Gene Expression Assays: PPIA (cyclophilin A; Hs99999904_m1), VCAN0 (Hs01007944_m1), VCAN1 (Hs01007937_m1), VCAN2 (Hs01007943_m1) and VCAN3 (Hs01007941_m1). Information about the alignments of the primers and the probes are publicly available at the manufacturers homepage www.appliedbiosystems.com using the TaqMan® Gene Expression Assay numbers listed above. Each reaction contained 10 μl of Gene Expression Master Mix, 1 μl of TaqMan Gene Expression Assay, 5 μl preamplified cDNA and 4 μl H₂O. Reactions were prepared in duplicate for each sample and incubated at 50° C. for 2 minutes, 95° C. for 10 minutes followed by 40 cycles of 95° C. for 15 seconds and 60° C. for 1 minute. The relative amounts of transcripts for each gene were normalised to the reference gene PPIA as follows: deltaC_{T}=C_{T} (gene of interest)−C_{T} (PPIA). The deltaC_{T} was linearized according to the formula 2^{-dCT} to determine the relative expression of each gene of interest.

Cell Culture.

For versican mRNA expression studies we used several cell lines of epithelial or mesenchymal origin: Renal proximal tubule cells derived from human (HK2) and pig (LLC-PK1), colon carcinoma cells (CACO-2), as well as human endothelial cells (EA.hy926) were purchased from American Type Culture Collection (ATCC). Primary proximal tubule cells (K2) were provided by Dr. C. Koppelstaetter (Department of Nephrology, Innsbruck Medical University, Austria). Immortalized prostate epithelial cells (EP156T, EP153T), primary smooth muscle cells (SMC), primary foreskin fibroblasts (VHF), and primary skin fibroblasts (HF) were obtained from Dr. Iris E. Eder at the Department of Urology from the Innsbruck Medical University. Real-time PCR of the versican isoforms was performed as described above, but the RNA was not pre-amplified. Ct values of versican and PPIA as assessed by ABI sequence detection software (version 1.3) were used to calculate the deltaCt using Microsoft Excel. Values are shown as ratio to the housekeeper PPIA (2 exp deltaCt).

Results I:

Identification of Versican Expression as Biomarker of Progressive Renal Disease.

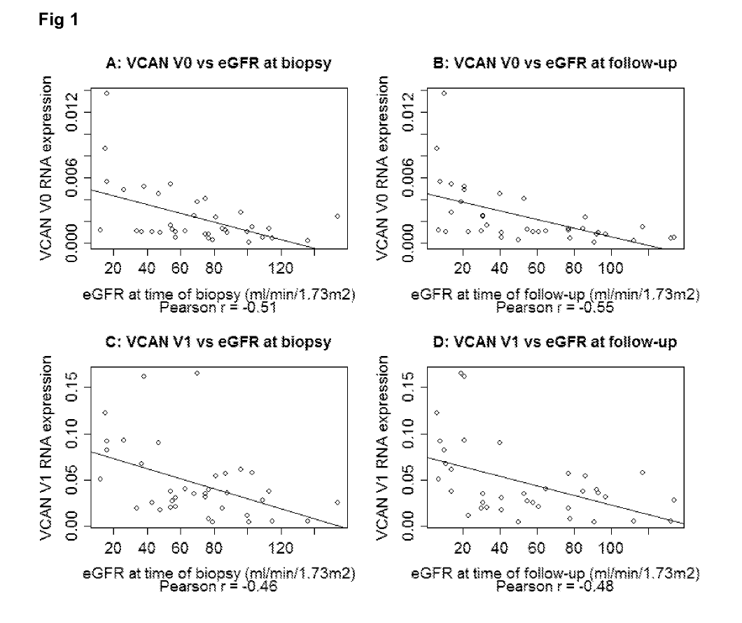

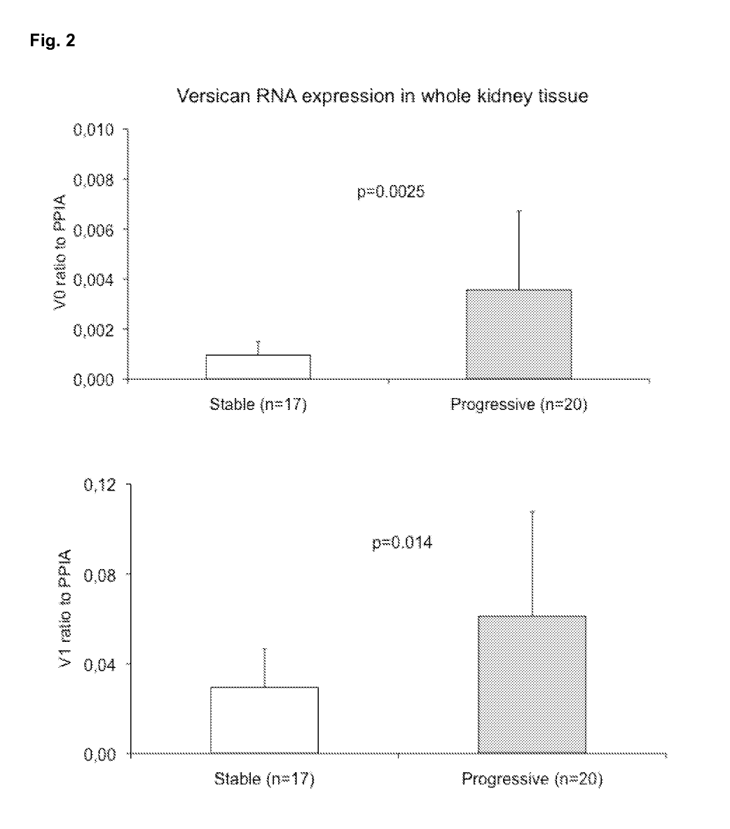

We evaluated the expression of versican isoforms V0, V1, V2 and V3 in an independent cohort of 37 patients with various proteinuric kidney diseases (Table 2). The expression of versican isoforms V0 and V1 showed a significant negative correlation with eGFR at time of biopsy and with eGFR at time of follow up (FIG. 1). We did not detect any expression of the isoform V2 in these samples. The versican isoform V3 showed a weak downregulation in subjects with lower eGFR, which was statistically significant (p=0.011) but clinically irrelevant (FIG. 2). Patients were classified as stable or progressive according to changes in eGFR during a median follow-up time of 25 months (2-80 months). As shown in FIG. 2, the expression of the isoforms V0 and V1 was significantly higher in progressive disease (V0: 3.7 fold, p=0.0025; V1: 2.1 fold, p=0.014). The V2 isoform was not expressed in these samples. The versican isoform V3 was downregulated in progressive patients by 2%. No significant correlation of versican expression to proteinuria, the degree of tubular atrophy and interstitial fibrosis nor the histological diagnosis could be detected. Linear regression analysis was performed for the different VCAN isoforms using the estimated GFR at follow up time as dependent variable. The expression of VCAN isoform 0 was negatively correlated with the estimated GFR (Pearson R=−0.54) and was the single most predictive VCAN isoforms explaining 27.7% (p-value<0.001) of the variability of the estimated GFR. The VCAN isoforms 1 explained 20.8% (p-value=0.002) of estimated GFR values at time of follow up. These results suggest a better predictive value of the versican isoforms V0 and V1 for progression of kidney disease, compared to established riskfactors such as degree of tubular atrophy and interstitial fibrosis and/or proteinuria.

TABLE 2
Patients included in the analysis of VCAN expression.
HD hemodialysis, NTX kidney transplantation. For abbreviation
of the histological diagnosis see Materials and Methods section.

| | | | | | | | | | | |
|---|---|---|---|---|---|---|---|---|---|---|
| | | | eGFR | Proteinuria | | | eGFR | Proteinuria | | |
| Subject | | Age | biopsy | biopsy | follow up time | | follow-up | follow-up | delta GFR | Histological |
| number | sex | (years) | (ml/min/m²) | (g/d) | (months) | ESRD | (ml/min/m²) | (g/d) | ml/min/year | diagnosis |

Stable disease

| | | | | | | | | | | |
|---|---|---|---|---|---|---|---|---|---|---|
| NC07 | m | 29 | 75 | 2.0 | 80 | — | 97 | 0.4 | 3.31 | IGAN |
| NC10 | m | 53 | 103 | 1.8 | 24 | — | 117 | 1.6 | 7.00 | IGAN |
| NC11 | m | 44 | 88 | 0.7 | 25 | — | 93 | 0.2 | 2.67 | IGAN |
| NC13 | m | 26 | 101 | 0.6 | 34 | — | 91 | 0.4 | −3.48 | IGAN |
| NC16 | m | 31 | 77 | 7.0 | 24 | — | 92 | 0.1 | 7.62 | MCN |
| NC17 | f | 56 | 109 | 17.0 | 30 | — | 134 | 0.0 | 9.72 | MCN |
| NC18 | m | 41 | 77 | 1.3 | 27 | — | 78 | 0.6 | 0.36 | MCN |
| NC19 | m | 69 | 57 | 8.0 | 24 | — | 61 | 0.2 | 2.18 | MCN |
| NC23 | m | 71 | 63 | 3.4 | 24 | — | 65 | 0.2 | 0.76 | MN |
| NC27 | f | 26 | 115 | 2.9 | 24 | — | 132 | 0.1 | 8.36 | pFSGS |
| NC43 | m | 31 | 55 | 4.4 | 25 | — | 55 | 2.2 | −0.15 | pFSGS |
| NC56 | f | 42 | 85 | 1.8 | 26 | — | 77 | 5.4 | −3.52 | pFSGS |
| NC70 | f | 31 | 113 | 5.8 | 25 | — | 85 | 3.7 | −13.53 | MCN |
| NC72 | m | 53 | 87 | 8.7 | 25 | — | 77 | 1.9 | −4.78 | MN |
| NC76 | f | 53 | 43 | 9.6 | 25 | — | 58 | 0.0 | 7.48 | MCN |
| NC81 | m | 20 | 136 | 2.2 | 12 | — | 112 | 0.1 | −23.06 | MCN |
| NC82 | f | 54 | 81 | 11.3 | 23 | — | 86 | 0.9 | 2.93 | MCN |

Progressive disease

| | | | | | | | | | | |
|---|---|---|---|---|---|---|---|---|---|---|
| NC01 | m | 51 | 12 | 7.2 | 5 | HD | 7 | 9.1 | −11.98 | DN |
| NC06 | m | 29 | 15 | 3.2 | 6 | NTX | 6 | 1.4 | −18.01 | IGAN |
| NC14 | f | 24 | 75 | 1.3 | 24 | — | 53 | 0.2 | −11.34 | IGAN |
| NC29 | m | 54 | 70 | 3.3 | 29 | — | 19 | 1.0 | −21.16 | DN |
| NC31 | m | 58 | 26 | 5.1 | 26 | — | 21 | 1.9 | −2.29 | HN |
| NC32 | f | 47 | 54 | 1.7 | 61 | — | 14 | 1.4 | −8.06 | HN |
| NC33 | m | 59 | 34 | 2.9 | 26 | — | 30 | 3.2 | −1.96 | U |
| NC34 | m | 41 | 16 | 3.0 | 2 | HD | 8 | 3.4 | −54.86 | IGAN |
| NC35 | m | 42 | 48 | 0.9 | 34 | — | 41 | 0.3 | −2.44 | IGAN |
| NC37 | m | 48 | 38 | 3.6 | 25 | — | 21 | 2.7 | −7.97 | IGAN |
| NC38 | m | 20 | 96 | 1.7 | 41 | — | 14 | 3.3 | −24.20 | IGAN |
| NC39 | f | 63 | 37 | 8.5 | 26 | — | 11 | 6.9 | −12.32 | MN |
| NC42 | f | 20 | 47 | 1.7 | 32 | — | 40 | 0.4 | −2.78 | pFSGS |
| NC44 | m | 43 | 57 | 4.5 | 26 | — | 41 | 5.4 | −7.50 | pFSGS |
| NC48 | m | 35 | 16 | 4.8 | 4 | HD | 10 | 2.0 | −15.42 | IGAN |
| NC50 | m | 71 | 54 | 3.6 | 21 | — | 33 | n.a. | −11.86 | pFSGS |
| NC51 | m | 51 | 100 | 1.3 | 26 | — | 23 | 7.6 | −36.09 | MN |
| NC52 | m | 71 | 68 | 2.5 | 25 | — | 31 | 2.4 | −18.05 | MN |
| NC73 | f | 69 | 79 | 4.8 | 27 | — | 50 | 1.2 | −12.89 | MN |
| NC89 | f | 63 | 154 | 3.0 | 22 | — | 31 | 1.0 | −65.68 | MN |

### Example 2

### VCAN mRNA Expression in Human Kidney Biopsies

Patients and Kidney Biopsies

We extended the Results I above and used kidney biopsies obtained from 74 patients with proteinuric renal diseases during their routine diagnostic workup for which we had complete clinical follow-up data (Table 3): diabetic nephropathy n=3, hypertensive nephropathy n=6, IgA nephropathy n=19, minimal change disease n=9, membranous nephropathy n=8, focal-segmental glomerulonephritis n=8, goodpasture syndrome n=2, interstitial nephritis n=4, lupus nephritis n=2, membranoproliferative glomerulonephritis n=2, ANCA-associated ANCA vasculitis n=6, rapid-progressive glomerulonephritis n=1, unknown and other n=4. The median follow-up time was 25 months (2-80). Based upon the estimated glomerular filtration rate (eGFR), which was calculated using the modified MDRD formula, patients were divided into a stable and a progressive cohort: Patients were defined stable when eGFR was >60 ml/min/1.73 m² at both timepoints, or when eGFR was <60 ml/min/1.73 m² at either timepoint and the decline in eGFR over time was >−1 ml/min/1.73 m². Patients were defined as progressive when eGFR was >60 ml/min/1.73 m² at time of biopsy and <60 ml/min/1.73 m² during follow-up, or when eGFR<60 ml/min/1.73 m² at both timepoints and delta eGFR was less than −1 ml/min/1.73 m², or when they reached end-stage renal disease. Tubular atrophy and interstitial fibrosis (TAIF) were scored by an independent pathologist following a semiquantitative grading system on haematoxylin/eosin and periodic-acid-Schiff- or Pearse-stained sections: none, mild (1-10%), moderate (11-30%), severe (>30%). The use of surplus material from routine biopsies (i.e. biopsy material, serum and urine) for gene expression profiling has been accredited by the Institutional Review Board of the Medical University of Innsbruck.

RNA Isolation and Real-Time PCR

Total RNA of whole kidney cryosections was isolated using the RNeasy® Micro Kit (Qiagen, Valencia Calif.). RNA was reverse transcribed into cDNA with the High Capacity cDNA reverse Transcription kit (Applied Biosystems, Foster City Calif.) in a 50 μl reaction according to the manufacturer's instructions. Preamplification was performed using TaqMan® Gene Expression Assays (vide infra) and the TaqMan® PreAmp Master Mix. Briefly, equal volumes of 20× TaqMan Gene Expression Assays were pooled and diluted to 0.2× with TE buffer. A 50 μl reaction containing 12.5 ul pooled assay mix, 25 μl TaqMan Preamp Master Mix and 5 ng of cDNA was prepared per sample and incubated in a thermocycler for 10 min at 95° C. followed by 10 cycles of 95° C. for 15 seconds and 60° C. for 4 minutes. Samples were then immediately cooled and diluted to 250 ul with TE buffer. All gene expression assays used had been previously tested to ensure uniform preamplification as recommended by the manufacturer.

The preamplified cDNA was analysed on the 7500 Fast Real-Time PCR System (Applied Biosystems) using the following inventoried human TaqMan® Gene Expression Assays: PPIA (cyclophilin A; Hs99999904_m1), VCAN0 (Hs01007944_m1), VCAN1 (Hs01007937_m1), VCAN2 (Hs01007943_m1) and VCAN3 (Hs01007941_m1). For real-time PCR experiments on RNA extracted from mouse tissue we used the following inventoried TaqMan® Gene Expression Assays: 18s (Hs03003631_g1), VCAN (Mm00490179_m1). Each reaction contained 10 μl of Gene Expression Master Mix, 1 μl of TaqMan Gene Expression Assay, 5 μl preamplified cDNA and 4 μl H₂O. Reactions were prepared in duplicate for each sample and incubated at 50° C. for 2 minutes, 95° C. for 10 minutes followed by 40 cycles of 95° C. for 15 seconds and 60° C. for 1 minute. The relative amounts of transcripts for each gene were normalised to the reference gene PPIA in human and 18s in mouse samples as follows: ΔC_{T}=C_{T} (gene of interest)−C_{T} (PPIA). The ΔC_{T} was linearized according to the formula 2^{-dCT} to determine the relative expression of each gene of interest.

Identification of Versican Expression as a Biomarker of Progressive Renal Disease

The expression of versican isoforms V0, V1, V2 and V3 was evaluated in an extended cohort of 74 patients with various proteinuric kidney diseases (Table 3). The expression of versican isoforms V0 and V1 showed a significant negative correlation with eGFR at time of biopsy and with eGFR at time of follow up: V0 vs eGFR biopsy r=−0.314 (p-value=0.003), V1 vs eGFR biopsy r=−0.303 (p-value=0.009), V0 vs eGFR follow-up r=−0.371 (p-value=0.0010) and V1 vs eGFR follow-up r=−0.385 (p-value=0.0007). We did not detect any expression of the isoform V2 in these samples. The versican isoform V3 did not show any correlation with eGFR. We did not detect any significant correlation of versican isoform expression with gender, age, proteinuria (biopsy and follow-up), histological diagnosis and the degree of tubular atrophy and interstitial fibrosis. The expression levels of the V1 isoform significantly correlated with the degree of interstitial inflammatory infiltrate (Kruskal Wallis test p-value: 0.014).

Patients were classified as stable or progressive according to changes in eGFR during a median follow-up time of 25 months (2-80 months). The expression of the isoforms V0 and V1 at time of biopsy was higher in patients with a progressive clinical course of the disease (V0: 1.7 fold, p=0.02; V1: 1.6 fold, p=0.05). The V2 isoform was not expressed in these samples. The versican isoform V3 did not show any difference in expression between stable and progressive patients.

TABLE 3
Patients included in the analysis of VCAN expression.
HD hemodialysis. NTX kidney transplantation. For abbreviation of the
histological diagnosis see text.

| | | | | | | | | | | |
|---|---|---|---|---|---|---|---|---|---|---|
| Subject | | Age | eGFR | Proteinuria | follow up | | eGFR | Proteinuria | delta GFR | Histological |
| number | sex | (years) | (ml/min/m²) | (g/d) | (months) | ESRD? | (ml/min/m²) | (g/d) | ml/min/year | diagnosis |

Stable disease

| | | | | | | | | | | |
|---|---|---|---|---|---|---|---|---|---|---|
| NC02 | f | 70 | 29 | 7.2 | 28 | — | 42 | 0.09 | 5.8 | DN |
| NC04 | m | 46 | 40 | 0.9 | 28 | — | 49 | 0.24 | 4.0 | HN |
| NC05 | m | 55 | 107 | 0.7 | 39 | — | 107 | unknown | 0.0 | HN |
| NC07 | m | 29 | 75 | 2.0 | 80 | — | 95 | 0.40 | 3.0 | IGAN |
| NC08 | f | 41 | 28 | 4.1 | 25 | — | 31 | 1.14 | 1.5 | IGAN |
| NC10 | m | 53 | 103 | 1.8 | 24 | — | 67 | 1.57 | −18.7 | IGAN |
| NC11 | m | 44 | 88 | 0.7 | 25 | — | 94 | 0.20 | 2.8 | IGAN |
| NC12 | m | 33 | 55 | 1.2 | 24 | — | 57 | 0.71 | 0.7 | IGAN |
| NC13 | m | 26 | 101 | 0.6 | 34 | — | 83 | 0.36 | −6.3 | IGAN |
| NC14 | f | 24 | 75 | 1.3 | 24 | — | 62 | 0.23 | −6.7 | IGAN |
| NC16 | m | 31 | 77 | 7.0 | 24 | — | 89 | 0.07 | 6.3 | MCD |
| NC17 | f | 56 | 81 | 17.0 | 30 | — | 61 | 0.02 | −7.9 | MCD |
| NC18 | m | 41 | 77 | 1.3 | 27 | — | 72 | 0.62 | −2.5 | MCD |
| NC19 | m | 69 | 57 | 8.0 | 24 | — | 81 | 0.23 | 11.9 | MCD |
| NC23 | m | 71 | 63 | 3.4 | 24 | — | 72 | 0.20 | 4.3 | MN |
| NC24 | f | 71 | 6 | 3.9 | 24 | — | 39 | 0.11 | 16.5 | IN |
| NC25 | m | 23 | 36 | 0.3 | 25 | — | 44 | 0.92 | 4.1 | IN |
| NC26 | f | 41 | 84 | 1.3 | 25 | — | 100 | 0.14 | 7.4 | RPGN |
| NC27 | f | 26 | 115 | 2.9 | 24 | — | 95 | 0.15 | −10.1 | pFSGS |
| NC50 | m | 71 | 54 | 3.6 | 21 | — | 58 | n.a. | 2.7 | pFSGS |
| NC53 | m | 49 | 81 | 1.4 | 25 | — | 70 | 11.18 | −5.1 | LN |
| NC54 | f | 51 | 22 | 3.8 | 24 | — | 35 | 2.90 | 6.6 | LN |
| NC55 | f | 64 | 28 | 10.3 | 31 | — | 43 | 0.30 | 6.0 | pFSGS |
| NC56 | f | 42 | 85 | 1.8 | 26 | — | 64 | 5.36 | −9.5 | pFSGS |
| NC57 | m | 24 | 53 | 0.6 | 24 | — | 77 | 0.04 | 12.2 | IGAN |
| NC58 | f | 59 | 35 | 3.3 | 25 | — | 47 | 0.53 | 5.4 | MPGN |
| NC59 | f | 24 | 10 | 0.7 | 25 | — | 71 | 0.72 | 29.7 | Goodpasture |
| NC60 | m | 29 | 19 | 0.4 | 24 | — | 48 | 0.42 | 14.2 | HN |
| NC62 | m | 37 | 104 | 1.9 | 27 | — | 92 | 1.96 | −5.0 | IGAN |
| NC63 | m | 20 | 67 | 10.8 | 28 | — | 103 | 0.62 | 15.4 | Goodpasture |
| NC64 | m | 81 | 20 | 0.4 | 25 | — | 29 | 0.17 | 4.6 | Vasculitis |
| NC65 | f | 53 | 59 | 0.6 | 25 | — | 58 | 0.26 | −0.3 | IGAN |
| NC66 | f | 72 | 24 | 2.5 | 24 | — | 58 | 0.00 | 16.8 | Vasculitis |
| NC67 | m | 60 | 49 | 0.1 | 21 | — | 64 | 0.07 | 8.7 | IN |
| NC68 | m | 37 | 19 | 0.6 | 24 | — | 72 | 0.17 | 26.1 | IGAN/Vasc. |
| NC69 | m | 49 | 55 | 0.0 | 22 | — | 91 | 0.00 | 20.1 | other |
| NC70 | f | 31 | 113 | 5.8 | 25 | — | 107 | 3.70 | −2.8 | MCD |
| NC72 | m | 53 | 87 | 8.7 | 25 | — | 80 | 1.94 | −3.7 | MN |
| NC73 | f | 69 | 79 | 4.8 | 27 | — | 68 | 1.18 | −4.9 | MN |
| NC74 | m | 68 | 25 | 0.2 | 26 | — | 45 | 2.28 | 8.9 | IGAN |
| NC75 | m | 74 | 59 | 5.9 | 27 | — | 65 | 0.00 | 2.7 | MN |
| NC76 | f | 53 | 43 | 9.6 | 25 | — | 67 | 0.00 | 12.1 | MCD |
| NC77 | m | 64 | 9 | 0.9 | 25 | — | 38 | 0.07 | 13.6 | Vasculitis |
| NC78 | m | 74 | 9 | 1.2 | 14 | — | 11 | 2.93 | 1.8 | HN |
| NC79 | f | 27 | 116 | 2.6 | 14 | — | 112 | NA | −4.1 | other |
| NC80 | m | 64 | 85 | 0.3 | 24 | — | 71 | 0.07 | −7.0 | Vasculitis |
| NC81 | m | 20 | 136 | 2.2 | 12 | — | 113 | 0.08 | −22.4 | MCD |
| NC82 | f | 54 | 81 | 11.3 | 23 | — | 81 | 0.94 | 0.2 | MCD |
| NC83 | m | 32 | 104 | 0.2 | 25 | — | 82 | 0.36 | −10.7 | Vasculitis |
| NC86 | f | 66 | 40 | 10.9 | 36 | — | 55 | 0.05 | 4.8 | MCD |
| NC88 | m | 58 | 80 | 0.3 | 13 | — | 70 | 0.07 | −8.9 | Vasculitis |
| NC89 | f | 63 | 154 | 3.0 | 22 | — | 120 | 1.50 | −17.9 | MN |

Progressive disease

| | | | | | | | | | | |
|---|---|---|---|---|---|---|---|---|---|---|
| NC01 | m | 51 | 12 | 7.2 | 5 | HD | 7 | 9.14 | −11.9 | DN |
| NC06 | m | 29 | 15 | 3.2 | 6 | NTX | 6 | 1.38 | −17.9 | IGAN |
| NC29 | m | 54 | 70 | 3.3 | 29 | — | 15 | 0.96 | −22.9 | DN |
| NC31 | m | 58 | 26 | 5.1 | 26 | — | 7 | 1.95 | −8.8 | HN |
| NC32 | f | 47 | 54 | 1.7 | 61 | — | 14 | 1.36 | −8.1 | HN |
| NC33 | m | 59 | 34 | 2.9 | 26 | — | 8 | 3.16 | −11.9 | unknown |
| NC34 | m | 41 | 16 | 3.0 | 2 | HD | 8 | 3.39 | −54.6 | IGAN |
| NC35 | m | 42 | 48 | 0.9 | 34 | — | 8 | 0.28 | −14.2 | IGAN |
| NC37 | m | 48 | 38 | 3.6 | 25 | — | 8 | 2.68 | −14.4 | IGAN |
| NC38 | m | 20 | 96 | 1.7 | 41 | — | 14 | 3.29 | −24.2 | IGAN |
| NC39 | f | 63 | 37 | 8.5 | 26 | — | 3 | 6.86 | −15.9 | MN |
| NC40 | m | 54 | 52 | 1.7 | 26 | — | 30 | 1.26 | −10.4 | MPGN |
| NC41 | m | 35 | 46 | 0.1 | 25 | — | 32 | 0.11 | −7.0 | IN |
| NC42 | f | 20 | 47 | 1.7 | 32 | — | 40 | 0.41 | −2.7 | pFSGS |
| NC43 | m | 31 | 55 | 4.4 | 25 | — | 44 | 2.21 | −5.4 | pFSGS |
| NC44 | m | 43 | 57 | 4.5 | 26 | — | 38 | 5.37 | −9.0 | pFSGS |
| NC45 | f | 64 | 30 | 3.9 | 26 | — | 9 | 0.24 | −9.6 | sFSGS |
| NC47 | m | 50 | 47 | 6.5 | 22 | — | 32 | 7.51 | −8.2 | IGAN |

Versican is Expressed in Renal Epithelial Cells and in Fibroblasts In Vitro.

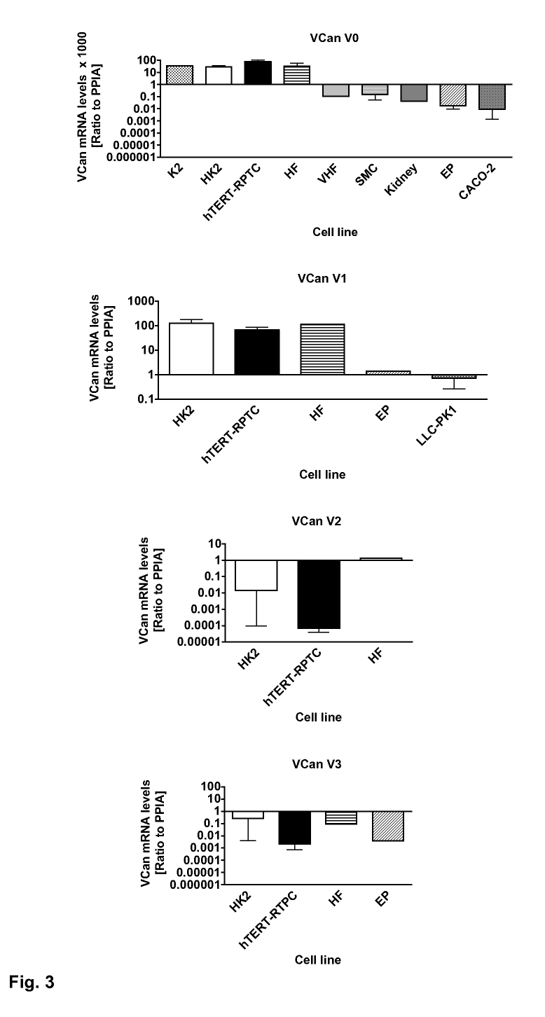

To analyze if versican expression is cell and/or organ specific we performed real-time PCR of the versican isoforms in cultured cells of epithelial and mesenchymal origin. We identified a massive basal expression of versican isoforms V0 and V1 in primary and immortalized human proximal tubule cells (FIG. 3) and in human skin fibroblasts. Other cells such as foreskin fibroblasts, smooth muscle cells, prostate epithelial cells and colon epithelial cells showed a versican expression which was 100-1000 times less than in the kidney epithelial cells. Interestingly, whole kidney tissues from healthy controls did not show V0 and V1 expression, also pointing towards the use of VCAN for diagnosing chronic kidney disease at early stage. The levels of versican isoform V2 were extremely low in all cell lines studied, in particular in all renal epithelial cells. Although we detected some differences in the expression of the versican isoform V3, the differences were not statistically significant between the cells lines. We did not detect any of the versican isoforms in human endothelial cells. These data suggest a cell specific and probably an organ specific expression of the versican isoforms, and they represent preliminary results which are the basis for further studies of versican expression and regulation in kidney cells.

Discussion

The novel biomarker candidates for identifying and monitoring progressive chronic kidney disease have the potential to predict the course of CKD already at an early stage when kidney function is close to normal or only slightly impaired. This information could be used to decide whether more aggressive therapies—stronger blood pressure lowering, higher doses of RAAS blockade, intensified immunosuppression—are of potential benefit for the individual patient. On the other hand the harms and benefits of such intensified therapeutic options should be carefully weighted in patients showing low biomarker expression levels thus having a potentially benign course of disease.

Using a bioinformatics analysis procedure of differential gene expression data we identified versican as a biomarker for histopathological damage in healthy kidneys, kidney grafts and in proteinuric kidney disease. In a second step we analysed the expression of versican in an independent cohort of 37 patients with various proteinuric kidney diseases and well-defined postbioptical clinical course with a median follow up time of 25 months (2-81 months; patients who were not on dialysis at end of follow-up had a follow-up time of 12-81 months). Two isoforms of versican (0 and 1) were significantly upregulated in those patients who showed a progressive loss of kidney function, suggesting that versican might serve as a potential predictive biomarker for progressive renal failure already at time of biopsy.

Versican is an extracellular matrix protein, which belongs to the family of hyaluronan-binding proteoglycans that include aggrecan, neurocan and brevican. These proteins have been grouped together on the basis of their structural similarity, and their ability to bind to the glycosaminoglycan (GAG) hyaluronan. This specific feature has also led to the collective term “hyalectins”. Each of the members shows a specific tissue distribution with aggrecan being mainly expressed in cartilage, and neurocan and brevican being confined to central nervous tissue. In contrast to these rather restricted expression patterns, versican appears to show a much wider tissue distribution with expression in a variety of soft tissues. The gene and protein structure of the hyalectins show highly conserved N- and C-terminal domains: The globular amino-terminal domain (G1) is responsible for binding to hyaluronan (sometimes called “hyaluronan binding region—HABR”), while the C-terminal domain resembles the selectin family of the proteins consisting of C-type lectin, two epidermal growth factor (EGF)-like domains and a complement regulatory region (often called the “EELC domain”, or G3 domain). The middle GAG binding region, however, shows little resemblance between the members of this family of proteins. While aggrecan contains up to 100 GAG side chains attached to this region, brevican, neurocan and versican contain only few chondroitin side chains. To date five isoforms of versican (V0, V1, V2, V3 and Vint) have been identified, which in most (V0, V1, V2, V3) but not all (Vint) cases result from alternative splicing of the two central exons 7 and 8 encoding the central glycosaminoglycan carrying regions, glycosaminoglycan alpha and beta (Dours-Zimmermann et al J Biol Chem 1994; 269: 32992-32998). The isoform V0 is the largest splice variant containing the N-terminal domain, both GAG-domains and the C-terminal EELC domain. The isoform V1 contains the GAG-beta but not the GAG-alpha and the isoform V2 contains the GAG-alpha but not the GAG-beta domain. The isoform V3 lacks both GAG domains resulting in no GAG attachement sites and therefore no GAG side chains. The size of the respective isoforms is predicted to be approximately 370 kDa for V0, 265 kDa for V1, 182 for V2 and 74 kDa for V3. Vint resembles an incomplete splice variant which probably retains the final intron in the carboxyterminal end of the protein. This isoform was identified by Lemire et al. (Lemire J M et al Arterioscler Thromb Vasc Biol 1999; 19: 1630-1639) and its characteristics as well as pathophysioloigical role are unclear.

Versican is expressed in healthy adult kidneys only at low levels. In chronic kidney disease increased versican expression is found in renal tissue with higher histopathological damage scores. Renal versican V0 or V1 mRNA expression is significantly higher in patients showing a progressive course of CKD than in patients with stable renal function. We demonstrated the propensity of this biomarker on the level of mRNA, indicating the propensity also on the level of protein.

### Example 3

### VCAN mRNA Expression in a Glomerulonephritis Mouse Model

Glomerulonephritis Mouse Model

Eight- to twelve-wk-old male C57Bl/6J mice obtained from Charles River (Sulzfeld, Germany) were used throughout the studies. Animals were maintained in a virus/antibody-free central animal facility of the Innsbruck Medical University. Accelerated anti-GBM nephritis was induced as described previously (Rosenkranz, J Clin Invest 103:649-659, 1999). In brief, mice were subcutaneously pre-immunized with 2 mg/ml rabbit IgG (Jackson ImmunoResearch Laboratories, West Grove, Pa.) dissolved in incomplete Freund's adjuvant (Sigma, St. Louis, Mich.) and nonviable desiccated Mycobacterium tuberculosis H37a (Difco Laboratories, Detroit, Mich.). After 5 d, heat-inactivated rabbit anti-mouse GBM antiserum was injected via the tail vein. All animal experiments were approved by Austrian veterinary authorities. The animals were sacrificed after 14 days, the kidneys were procured and RNA was extracted as stated above.

Versican as a Marker of Renal Injury in the Glomerulonephritis Mouse Model

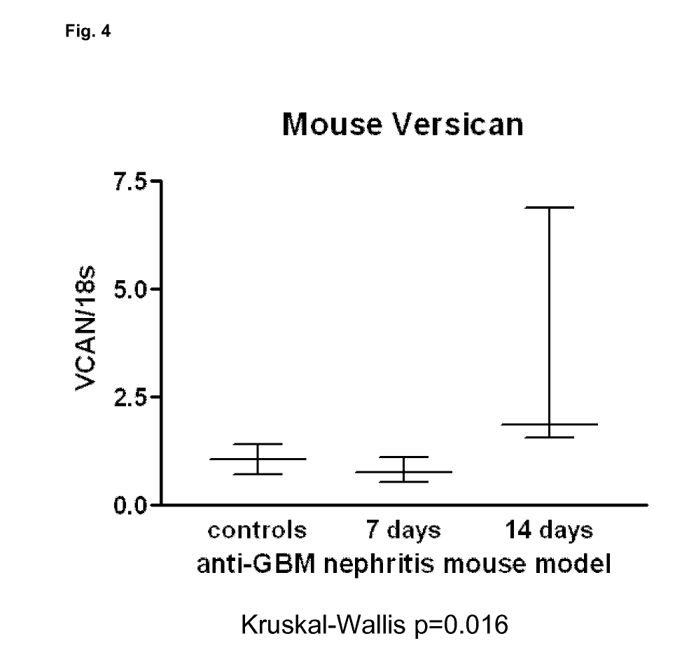

The resulting nephrotoxic nephritis is characterized by significant proteinuria but only slight creatinine elevation. Histological changes consisted of focal mesangial hypercellularity, focal and mild deposits of PAS⁺ hyaline material in lumina and increases in mesangial matrix occurring in less than 10% of glomeruli, and a mild focal interstitial mononuclear cell infiltrate. We analysed the expression of mouse Versican at day 0 (controls), after 7 and after 14 days. We did not detect any significant Versican upregulation after 7 days, but there was a strong and significant upregulation of Versican after 14 days (Kruskal Wallis test p-value: 0.016) (FIG. 4).

### Example 4

### VCAN Protein Expression in Human Renal Biopsies

Immunohistochemistry and Immunofluorescence

Frozen sections of representative stable and progressive subjects were stained for human Versican protein. The sections were fixed in cold acetone and incubated at room temperature for 60 min with a 1:400 dilution of the primary antibody (rabbit anti-human Versican, Santa Cruz, sc-25831, Santa Cruz, Calif., USA). Versican was detected by the Vectastain Elite ABC Kit (Vector Laboratories, Burlingame, Calif., USA, www.vectorlabs.com) and stained with 3-amino-9-ethylcarbazole. This system uses a biotin-conjugated secondary antibody (1:1000), avidin and biotinylated horseradish peroxidase and the corresponding chromogen for visualization. All sections were counterstained with 3,30-diaminobenzidine tetrahydrochloride 3-amino-9-ethyl carbazole.

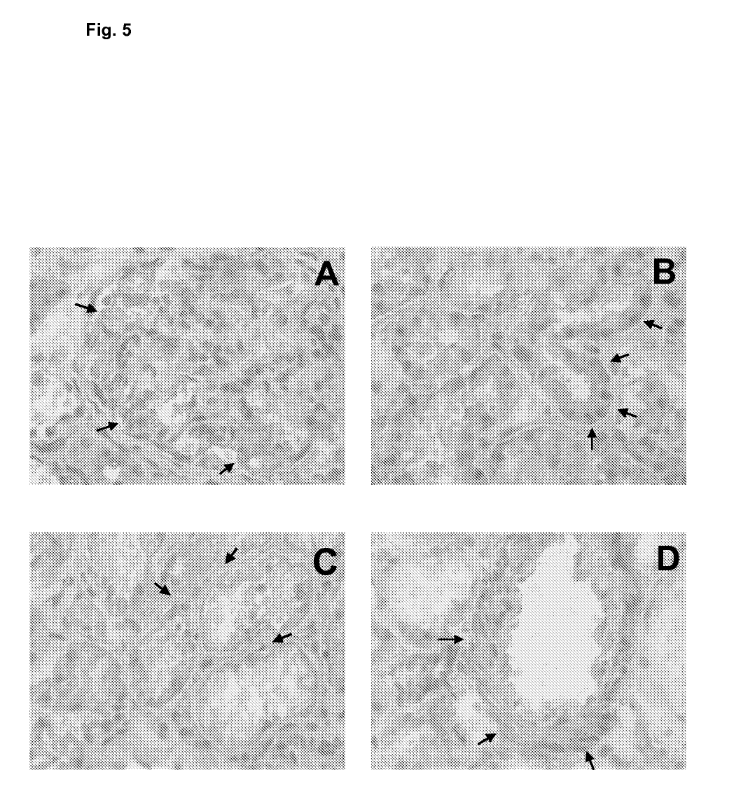

Versican protein was expressed in several compartments of the kidney biopsies. The weakest expression was found in the glomeruli (FIG. 5 A), while the strongest expression was found in the tubulinterstitial compartment, both in tubuli (FIG. 5 B) and in the interstitial fibroblasts and in areas of fibrosis (FIG. 5 C). Interestingly, Versican was also expressed in the media of some but not all renal cortical blood vessels (FIG. 5 D).

Versican, thus, qualifies as a marker of renal disorders. The differentiation between the versican isoforms and the specific determination of the inventive V0 and/or V1 will improve the determination of the risk of renal disorders, including the determination of renal disease.

1. An in vitro method of determining the risk of one or more renal disorders in a patient by measuring a versican (VCAN) parameter, characterized in that a kidney tissue sample of said patient is contacted with a solid phase binding assay to determine an amount of at least one of the isoforms V0 and V1 in the sample, and the amount is then compared to a predetermined reference level of V0 and/or V1.

2. The method according to claim 1, wherein both isoforms are determined.

3. The method according to claim 1, wherein said renal disorders are selected from the group consisting of acute kidney disease, chronic kidney disease, proteinuric kidney disease and progressive kidney disease.

4. The method according to claim 1, wherein the risk of one or more renal disorders in the patient is determined when the amount of said parameter is increased at least 1.5 times the reference value of subjects not at risk of the renal disorder.

5-6. (canceled)

7. The method according to claim 1, wherein the level of VCAN expression is determined.

8. The method according to claim 1, wherein VCAN nucleic acid and/or protein expression is determined.

9. The method according to claim 1, wherein said parameter is determined by a method selected from the group consisting of microarray hybridization with specific probes and PCR.

10. The method according to claim 1, wherein an additional kidney risk factor (KRF) or senescence parameter is measured, the additional parameter being a marker selected from the group consisting of IL1 RN, ISG15, LIFR, C6, IL32, NRP1, CCL2, CCL19, COL3A1, and GZMM.

11. The method according to claim 1, comprising the steps of:
(a) contacting a sample obtained from said patient with oligonucleotides that specifically hybridize to the V0 and/or V1 isoforms, and
(b) detecting in the sample a level of one or more polynucleotides that hybridize to the V0 and/or V1 isoforms and comparing said level relative to a predetermined cut-off value for each polynucleotide, and therefrom determining the risk of a renal disorder in the patient.

12. The method according to claim 1, comprising the step of:
comparing the levels of the V0 and/or V1 isoforms in a sample from said patient with normal levels of said isoforms in samples of the same type obtained from control patients, wherein altered levels of either isoform relative to the corresponding normal levels of the same isoform is an indication that the patient has a risk of a renal disorder.

13. The method according to claim 1, wherein the step of measuring a VCAN parameter comprises the step of quantitating the V0 and/or V1 isoforms in a sample from said patient by a method comprising
(a) reacting the sample with one or more binding agents specific for either one of the isoforms, said isoforms having been labeled with a detectable substance, and
(b) detecting the detectable substance.

14. The method according to claim 1, comprising the steps of:
(a) maintaining separate aliquots of a sample from a patient in the presence and absence of a test compound, and
(b) comparing the levels of the V0 and/or V1 isoforms in each of the aliquots maintained in the presence of the test compound to the aliquots maintained in the absence of the test compound.

15-16. (canceled)

17. A method for the diagnosis of renal disease in a patient, comprising:
measuring expression of a versican V0 isoform and/or a versican V1 isoform in a sample of the patient;
comparing the measured expression of at least one of the versican V0 and V1 isoforms to a predetermined reference level, wherein an increase in the expression of the isoforms V0 and/or V1 in the patient as compared to the reference level indicates that the patient is at risk of disease progression.

18. The method of claim 17, wherein measuring a VCAN parameter comprises the use of a set of reagents selected from the group consisting of:
a reagent which specifically binds to the VCAN V0 polypeptide, and
a reagent which specifically binds to the VCAN V1 polypeptide.

19. The method of claim 18, wherein said reagents are antibodies or antibody fragments.

20. The method of claim 18, wherein said reagents are labelled labeled.

21. The method according to claim 1, wherein an additional parameter is measured, the additional parameter being a marker selected from the group consisting of CDKN2A, CDKN1A, sirtiuns 1-8, XRCC5, G22P1, hPOT 1, collagenase, TANK 1, TANK 2, TRF 1, TRF 2, and WRN.


SEQUENCE LISTING (3 sequences)

SEQ ID NO: 1 - protein, 3396 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 2 - DNA, 12057 bp, Homo sapiens
acagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacctaaagaggctacaaccatcgatttgagtatcctcgcagaaactgcatcacccagtttatccaaagaaccacaaatggtttctgatagaactacaccaatcatccctttagttgatgaattacctgtcattccaacagagttccctcccgtgggaaatattgtcagttttgaacagaaagccacagtccaacctcaggctatcacagatagtttagccaccaaattacccacacctactggcagtaccaagaagccctgggatatggatgactactcaccttctgcttcaggacctcttggaaagctagacatatcagaaattaaggaagaagtgctccagagtacaactggcgtctctcattatgctacggattcatgggatggtgtcgtggaagataaacaaacacaagaatcggttacacagattgaacaaatagaagtgggtcctttggtaacatctatggaaatcttaaagcacattccttccaaggaattccctgtaactgaaacaccattggtaactgcaagaatgatcctggaatccaaaactgaaaagaaaatggtaagcactgtttctgaattggtaaccacaggtcactatggattcaccttgggagaagaggatgatgaagacagaacacttacagttggatctgatgagagcaccttgatctttgaccaaattcctgaagtcattacggtgtcaaagacttcagaagacaccatccacactcatttagaagacttggagtcagtctcagcatccacaactgtttcccctttaattatgcctgataataatggatcatccatggatgactgggaagagagacaaactagtggtaggataacggaagagtttcttggcaaatatctgtctactacaccttttccatcacagcatcgtacagaaatagaattgtttccttattctggtgataaaatattagtagagggaatttccacagttatttatccttctctacaaacagaaatgacacatagaagagaaagaacagaaacactaataccagagatgagaacagatacttatacagatgaaatacaagaagagatcactaaaagtccatttatgggaaaaacagaagaagaagtcttctctgggatgaaactctctacatctctctcagagccaattcatgttacagagtcttctgtggaaatgaccaagtcttttgatttcccaacattgataacaaagttaagtgcagagccaacagaagtaagagatatggaggaagactttacagcaactccaggtactacaaaatatgatgaaaatattacaacagtgcttttggcccatggtactttaagtgttgaagcagccactgtatcaaaatggtcatgggatgaagataatacaacatccaagcctttagagtctacagaaccttcagcctcttcaaaattgccccctgccttactcacaactgtggggatgaatggaaaggataaagacatcccaagtttcactgaagatggagcagatgaatttactcttattccagatagtactcaaaagcagttagaggaggttactgatgaagacatagcagcccatggaaaattcacaattagatttcagccaactacatcaactggtattgcagaaaagtcaactttgagagattctacaactgaagaaaaagttccacctatcacaagcactgaaggccaagtttatgcaaccatggaaggaagtgctttgggtgaagtagaagatgtggacctctctaagccagtatctactgttccccaatttgcacacacttcagaggtggaaggattagcatttgttagttatagtagcacccaagagcctactacttatgtagactcttcccataccattcctctttctgtaattcccaagacagactggggagtgttagtaccttctgttccatcagaagatgaagttctaggtgaaccctctcaagacatacttgtcattgatcagactcgccttgaagcgactatttctccagaaactatgagaacaacaaaaatcacagagggaacaactcaggaagaattcccttggaaagaacagactgcagagaaaccagttcctgctctcagttctacagcttggactcccaaggaggcagtaacaccactggatgaacaagagggcgatggatcagcatatacagtctctgaagatgaattgttgacaggttctgagagggtcccagttttagaaacaactccagttggaaaaattgatcacagtgtgtcttatccaccaggtgctgtaactgagcacaaagtgaaaacagatgaagtggtaacactaacaccacgcattgggccaaaagtatctttaagtccagggcctgaacaaaaatatgaaacagaaggtagtagtacaacaggatttacatcatctttgagtccttttagtacccacattacccagcttatggaagaaaccactactgagaaaacatccctagaggatattgatttaggctcaggattatttgaaaagcccaaagccacagaactcatagaattttcaacaatcaaagtcacagttccaagtgatattaccactgccttcagttcagtagacagacttcacacaacttcagcattcaagccatcttccgcgatcactaagaaaccacctctcatcgacagggaacctggtgaagaaacaaccagtgacatggtaatcattggagaatcaacatctcatgttcctcccactacccttgaagatattgtagccaaggaaacagaaaccgatattgatagagagtatttcacgacttcaagtcctcctgctacacagccaacaagaccacccactgtggaagacaaagaggcctttggacctcaggcgctttctacgccacagcccccagcaagcacaaaatttcaccctgacattaatgtttatattattgaggtcagagaaaataagacaggtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcgactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagtcgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggggattgttaaaacacatgcaaaaaaaaaaaaaaaaaaaaaaaaagaaattttgtatatataaccattttaatcttttataaagttttgaatgttcatgtatgaatgctgcagctgtgaagcatacataaataaatgaagtaagccatactgatttaatttattggatgttattttccctaagacctgaaaatgaacatagtatgctagttatttttcagtgttagccttttactttcctcacacaatttggaatcatataatataggtactttgtccctgattaaataatgtgacggatagaatgcatcaagtgtttattatgaaaagagtggaaaagtatatagcttttagcaaaaggtgtttgcccattctaagaaatgagcgaatatatagaaatagtgtgggcatttcttcctgttaggtggagtgtatgtgttgacatttctccccatctcttcccactctgttttctccccattatttgaataaagtgactgctgaagatgactttgaatccttatccacttaatttaatgtttaaagaaaaacctgtaatggaaagtaagactccttccctaatttcagtttagagcaacttgaagaagagtagacaaaaaataaaatgcacatagaaaaagagaaaaagggcacaaagggattggcccaatattgattctttttt

SEQ ID NO: 3 - protein, 2409 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKRRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR